(19) United States
(12) Patent Application Publication
(10) Pub. No.: US 2021/0228683 A1
(43) Pub. Date: Jul. 29, 2021

(54) WHEY PROTEIN CONCENTRATE IN ASSOCIATION WITH ANTITUMOUR TREATMENT

(71) Applicant: LB LYOPHARM S.R.L., Bolzano (IT)

(72) Inventor: Christian REBIERE, Bolzano (IT)

(73) Assignee: LB LYOPHARM S.R.L., Bolzano (IT)

(21) Appl. No.: 17/058,854

(22) PCT Filed: May 21, 2019
(86) PCT No.: PCT/IB2019/054180

§ 371 Date: Nov. 25, 2020

(30) Foreign Application Priority Data
May 25, 2018 (IT) 102018000005714

(51) Int. Cl. A61K 38/17; A61K 38/38; A61K 31/7016; A61K 39/395; A61P 3/02; A61K 45/06; A61P 7/00; A61P 1/00

(52) U.S. Cl. CPC A61K 38/1709; A61K 38/38; A61K 31/7016; A61K 39/395; A61N 5/10; A61K 45/06; A61P 7/00; A61P 1/00; A61K 38/385; A61P 3/02

(57) ABSTRACT

The present invention relates to highly purified whey proteins of well-defined composition for use in an anti-tumour therapy in combination with one or more anti-tumour drugs and/or radiotherapy.

The present invention relates to a highly purified whey protein of well-defined composition for use in an anti-tumour therapy in combination with one or more anti-tumour drugs and/or radiotherapy.

### PRIOR ART

Doctors and health workers in all sectors, in particular in the field of oncology, are advised to pay attention to the side effects caused or aggravated by pharmacological therapeutic treatment. In the case of oncology patients, one of the major problems is constituted by malnutrition, in particular sarcopenia and neoplastic cachexia, which is a problem that for some time has been completely overlooked in the medical field, and by toxicity, for example haematological, gastrointestinal and/or neurological.

The term sarcopenia was coined for the first time in 1997 by Irwin H. Rosenberg, from the University of Boston, who used it to describe the condition of age-related loss of muscular mass and function. More recently, EWGSOP (European Working Group on Sarcopenia in Older People) has provided a more precise working definition of sarcopenia, which is now described as a syndrome characterised by a progressive and generalised loss of musculoskeletal mass and strength, which can lead to a high risk of adverse events, including physical disability, low quality of life, and death.

The prevalence of sarcopenia in patients suffering from neoplastic disease is an extremely widespread phenomenon, and its incidence varies with the type of tumour in question and patient characteristics.

Changes to muscular mass can also be observed can be observed also as a direct consequence of the surgical, chemotherapeutic, immunotherapeutic and radiotherapeutic treatments to which oncology patients are subjected. These treatments, which often result in classified episodes of anorexia, an early feeling of satiety, nausea, vomiting, and changes in mood and sense of smell within the above-discussed scope of malnutrition, contribute to protein, and therefore also muscular depletion in the patient suffering from neoplastic disease.

There is ample evidence in the literature of the close relationship between body composition and prognosis in cancer patients. The condition of sarcopenia in an adult patient suffering from neoplasia must be considered to be an extremely negative prognostic factor, since it is associated with an increase in the toxicity risk of chemotherapy, has a greater frequency of post-surgery complications, and is associated with a reduction in the overall survival (OS). A study in 2015 (TAN BHL. et al. (2015) Sarcopenia is associated with toxicity in patients undergoing neo-adjiuvant chemotherapy for oesophago-gastric cancer.” EJSO, 41: 333-338) carried out on patients suffering from neoplasia of the oesophageal and gastric tracts, revealed how the presence of sarcopenia prior to the onset of neo-adjuvant chemotherapy treatment must be considered as a negative prognostic factor, since it is associated with a statistically significant reduction of the OS compared to non-sarcopenic patients.

In addition to malnutrition defined as sarcopenia, oncology patients also suffer from cachexia, which is a clinical syndrome frequently encountered in patients suffering from severe chronic cardiac, renal, pulmonary, infectious, neurological and neoplastic diseases; it is a serious condition, but is very often unrecognised and therefore is only identified or diagnosed late. Cachexia is nowadays defined as being a multifactorial syndrome characterised by a progressive loss of muscular mass (in association with or without loss of fatty mass) which cannot be fully corrected by conventional nutritional support and leads to an increase in functional damage. Cachexia is a complication that is very common in neoplastic disease: it is estimated that 70% of oncology patients will develop cachexia over time and that 20% of those will die as a result of complications associated with it. The incidence is very high and varies depending on the type of tumour: estimations are up to 80% of patients with gastric or pancreatic neoplasia, around 50% of subjects with pulmonary, prostate and colon-rectal tumours, and approximately 40% of women with breast neoplasia.

Once it has been determined that the patient requires nutritional intervention because they are already malnourished or are at risk of malnutrition, the best way to intervene will be defined on the basis of a consultation with a multidisciplinary team of experts including at least one nutritionist and oncologist.

Pharmacological approaches, which themselves are not devoid of side effects, such as the use of progestins, corticosteroids, antiemetics, and antidepressants are currently used. However, it is desirable, in particular in patients who are already undergoing or who will undergo highly debilitating therapies, to find solutions to the malnutrition and other toxicity-related side effects of anti-tumour drugs which use natural substances capable of improving the quality of daily life of patients, rather than other drugs that in turn have other side effects.

Numerous approaches have been adopted to identify suitable nutritional supplements which, when administered in combination with anti-tumour drugs, will succeed in reducing or preventing the development of side effects of anti-tumour drugs, such as malnutrition and toxicity of the drug on the patient to whom said drugs have been administered.

The literature describes approaches that comprise combination methods in which the supplement to be combined with the anti-tumour drug is administered orally, enterally, or parenterally. The latter two administration methods for the supplement to be combined with the anti-tumour drug have resulted in serious problems in respect of patient safety, for example infections and even sepsis.

Compounds such as proteins, vitamins, minerals, antioxidants, omega 3 fatty acids, amino acids and fibres, such as whey proteins or branched-chain amino acids, have been used for oral administration.

Whey proteins were used in a randomised double-blind versus placebo study (Rondanelli M., Klersy C., et al. (2016). “Whey protein, aminoacids, and vitamin D supplementation with physical activity increases fat-free mass and strength, functionality and quality of life and decreases inflammation in sarcopenic elderly.” American J. of Clin. Nutr., 103: 830-840), performed on patients suffering from primary sarcopenia, which demonstrated how their use, in combination with amino acids, vitamin D and with suitable physical activity, was effective not only in sustaining muscular mass and strength, but also in promoting a general improvement of well-being in elderly patients suffering from sarcopenia, with a statistically significant difference compared to that observed among the controls, who were subjected only to increased physical activity.

However, the literature does not present any data making it possible to identify a fixed link between possible forms of dietary supplements and anti-tumour drugs that significantly reduces some of the side effects of the anti-tumour drugs and that reduces or even prevents aggravation or development of malnutrition in oncology patients subjected to therapy with anti-tumour drugs.

### SUMMARY OF THE INVENTION

The authors of the present invention have surprisingly discovered that the use of a whey protein concentrate having a well-defined composition as a supplement administered to oncology patients subjected to anti-tumour treatment, by means of radiotherapy and/or anti-tumour drugs, reduces or even prevents the emergence of side effects associated with anti-tumour treatment that are observed in oncology patients subjected to the same treatment but without administration of said proteins as supplement.

The invention therefore relates to a whey protein concentrate for use in anti-tumour therapy in combination with one or more anti-tumour drugs and/or radiotherapy, wherein said whey protein concentrate has the following composition

| | | |
|---|---|---|
| Alpha-lactalbumin | 20-28% | by weight |
| Beta-lactalbumin | 32-45% | by weight |
| Seroalbumin | 5.9-7.8% | by weight |
| Immunoglobulins | 3-6% | by weight |
| K Casein | 4-7% | by weight |
| Beta casein | 2-5% | by weight |
| Casein alpha | 2-5% | by weight |
| Lactose | <1% | by weight |
| Fat content | 0% | by weight. |

In jurisdictions where this is permitted, the invention also relates to a therapeutic method for treating oncology patients comprising the administration of a whey protein concentrate to patients subjected to anti-tumour treatment by means of one or more anti-tumour drugs and/or radiotherapy,

wherein said whey protein concentrate has the following composition

| | | |
|---|---|---|
| Alpha-lactalbumin | 20-28% | by weight |
| Beta-lactalbumin | 32-45% | by weight |
| Seroalbumin | 5.9-7.8% | by weight |
| Immunoglobulins | 3-6% | by weight |
| K Casein | 4-7% | by weight |
| Beta casein | 2-5% | by weight |
| Casein alpha | 2-5% | by weight |
| Lactose | <1% | by weight |
| Fat content | 0% | by weight. |

### DETAILED DESCRIPTION OF THE FIGURES

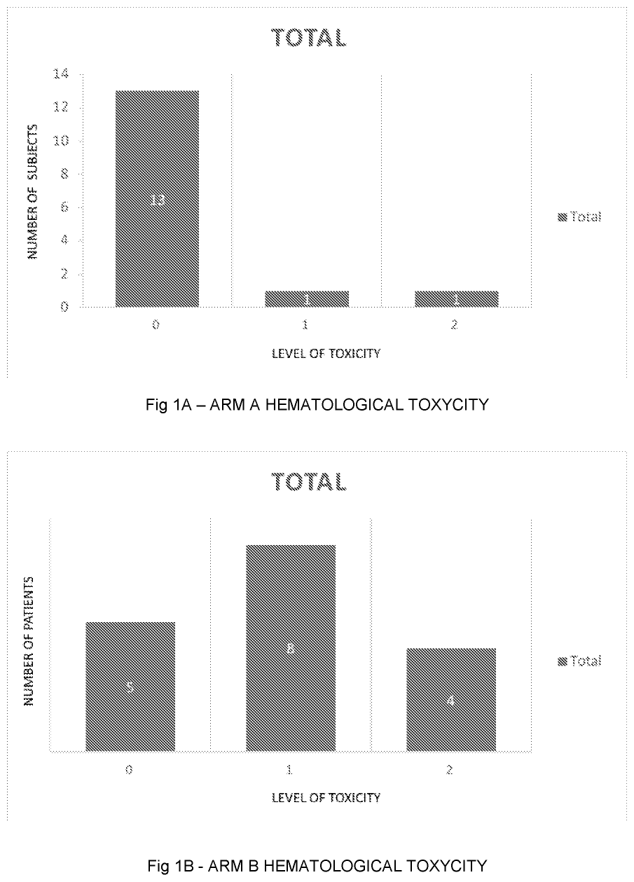

FIG. 1 shows the data for assessment of haematological toxicity (anaemia, leukopenia, thrombocytopenia) following chemotherapeutic treatment over a period of three months. Box 1A shows that 86% of patients from group A did not present any type of toxicity associated with the treatment, 6% reported grade 1 toxicity (1 subject) and 6%≥grade 2 toxicity (1 subject); box B shows that the difference in relation to the control group is considerable, since 71% of these patients demonstrated toxicity, whereas only 29% did not show any kind of adverse effect of haematological nature. This difference is statistically significant with a p value=0.005.

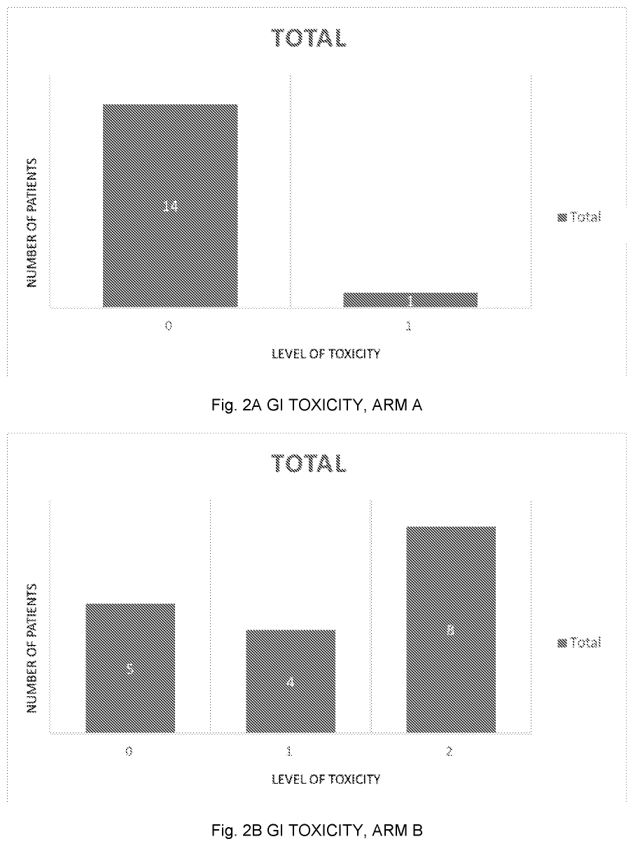

FIG. 2 shows the data for assessment of gastrointestinal (GI) toxicity (nausea, vomiting, diarrhoea, mucositis): Box A shows that 94% of patients from group A who took the proteins did not present any type of toxicity following the first three months of chemotherapeutic treatment, only 1 subject (6%) presented grade 1 toxicity, and no patients in the active group had≥grade 2 toxicity. Box B shows that in the group who took the placebo the distribution of the values is quite different: only 20% of patients did not present toxicity, whereas 70% presented adverse effects. This difference is statistically significant with a p value=0.001.

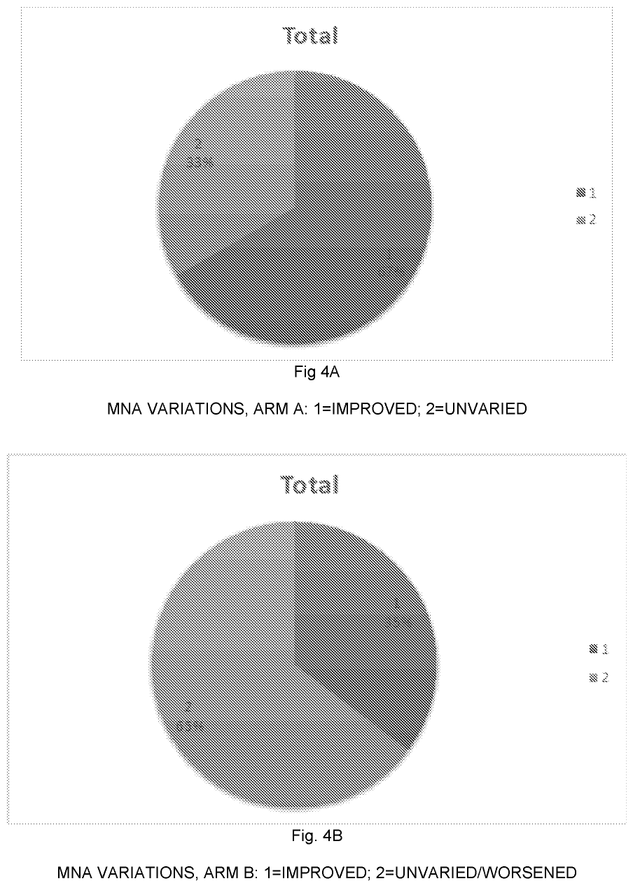

FIG. 4 shows the differences between groups A and B in the clinical test in terms of MNA (Mini Nutritional Assessment) variation, and body weight distribution. Box A shows the MNA variations in group A, in which 66% of patients presented an improvement in the score, whereas box B shows that in group B instead 64% of patients remained stable or even deteriorated.

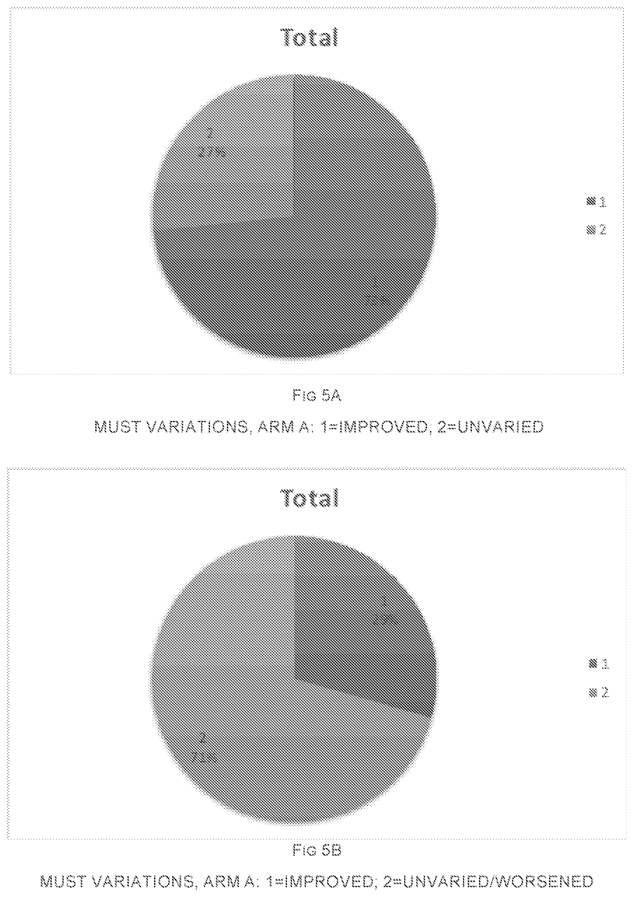

FIG. 5 shows the differences between groups A and B in the clinical test in terms of MUST (Malnutrition Universal Screening Tool) variations. Box A shows that 74% of patients in the active group presented an improved score after three months of treatment, whereas box B shows that in 71% of patients in the placebo group a stability or a deterioration was observed. This difference is statistically significant (p value=0.013), the RR is 0.15 (95%IC=0.03-0.71), defining protein integration as a protective factor of 85% compared to the observance of stability or deterioration of the nutritional risk of the patient according to MUST.

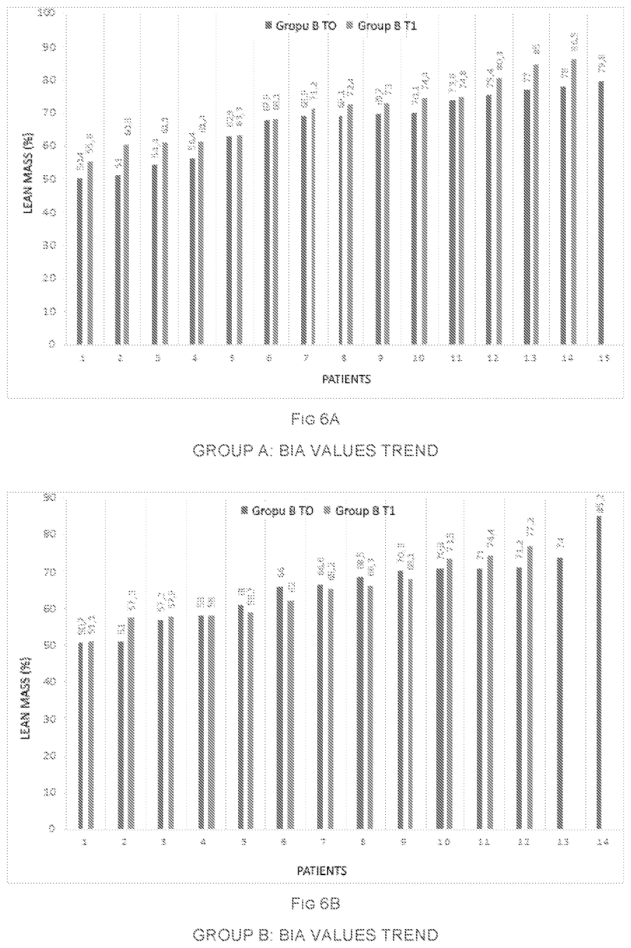

FIG. 6 shows the differences between groups A and B in the clinical test in terms of bioimpedance findings, which reveal (box A) that the lean mass of the patients in group A is increased compared to the measurement over time TO (71.8% vs 69.7%), whereas that of the subjects in group B (box B) decreases from 67.6% to 63.6%. The variations in fat mass reveal a similar, complementary favourable trend. The variations observed after three months in the patients in the active group is statistically significant (p value=0.013, Z=-2.480).

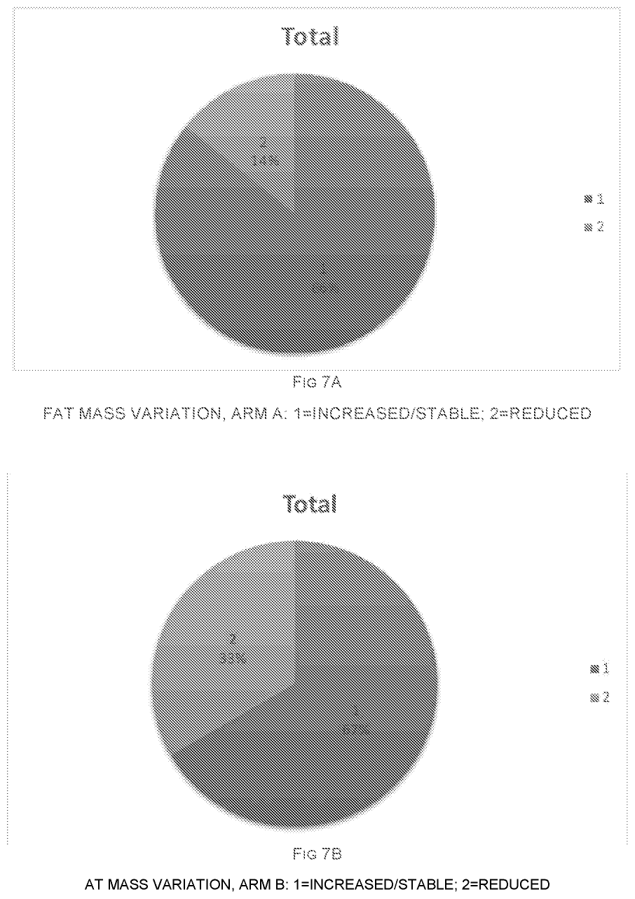

FIG. 7 shows the patients with stable or increased fat mass values over time, and patients with reduced mass in the two groups of the clinical test. Box A shows that in group A 86% of the patients have maintained a stable lean mass or have experienced increased lean mass over the course of time, whereas 33% of the patients in group B experienced a reduction of lean mass. This difference is not statistically significant, and the RR is 0.33 (95% IC=0.05-2.27), and therefore the administration of the protein supplement reduces the risk of lean mass reduction by 67%.

### GLOSSARY

The term “lactoserum” or “whey” in the present description means the liquid part obtained from whole milk, skimmed milk, or semi-skimmed milk after separation of the rennet.

The expression “milk whey proteins” means those proteins that in the literature are also known as whey proteins or WPIs (“whey proteins isolate” or also “lactoserum concentrate” or “whey protein concentrate”, the last two terms being used as synonyms for “whey protein” throughout the present description and in the claims). The meaning of the term is also clear from the information presented in the present description.

The term “sarcopenia” in the present description means the pathological condition which manifests itself in the form of the onset of a loss of muscular mass as defined in the literature and in the above discussion of the prior art.

The term “cachexia” or wasting syndrome in the present description means a loss of weight, muscular atrophy, tiredness, weakness and significant loss of appetite not caused by anorexia. The formal definition of cachexia is a loss of body weight that cannot be reversed nutritionally: even if the individual suffering from the affliction were to consume more calories, the lean body mass would still decrease, thus indicating the presence of a primary pathology, as defined in the literature and in the above discussion of the prior art. The term as used in the present description (and disclosed also in the above discussion of the prior art) corresponds to the use of said term in the literature.

### DETAILED DESCRIPTION OF THE INVENTION

As stated in the summary above, the authors of the invention have surprisingly found that supplementation with a concentrate of highly purified whey protein of well-defined composition, in addition to preventing or improving the general condition of oncology patients in terms of their nutritional state (as described in detail in the examples below and as shown in the figures) and preventing any aggravation of the nutritional state of the oncology patients subjected to anti-tumour therapy, also reduced the haematological and gastrointestinal toxicity of anti-tumour therapy to practically zero in patients taking said concentrate, during the anti-tumour therapy, and therefore in combination therewith, said concentrate being a whey protein concentrate having the following composition:

| | | |
|---|---|---|
| Alpha-lactalbumin | 20-28% | by weight |
| Beta-lactalbumin | 32-45% | by weight |
| Seroalbumin | 5.9-7.8% | by weight |
| Immunoglobulins | 3-6% | by weight |
| K Casein | 4-7% | by weight |
| Beta casein | 2-5% | by weight |
| Casein alpha | 2-5% | by weight |
| Lactose | <1% | by weight |
| Fat content | 0% | by weight. |

In one embodiment, in the above-mentioned proteins, the lactoserum is present in a concentration of <0.5%, also <0.1%, or also or even <0.05% (that is to say≤or also <0.5 grams per kilo of protein).

The composition as provided is based on proteins in lyophilised form.

Such proteins, other than in terms of composition, also differ from the currently commercially available proteins not produced by the applicant in tests on cells. The authors of the invention have in fact surprisingly found that the addition of the concentrate of whey proteins according to the invention to cellular culture media is not toxic for the cells, whereas equal quantities of whey protein concentrates which are not the concentrate produced by the applicant of the present application are toxic for the cells (example 2).

The authors of the invention have found that the whey protein concentrate of the above-mentioned composition improved or prevented a deterioration of the nutritional state of patients consuming such proteins daily during the prior of time within which they were subjected to anti-tumour treatment compared to patients subjected to the same anti-tumour treatment but not consuming the aforementioned proteins.

In addition, the consumption of the proteins with the above-described composition led to a statistically significant reduction of haematological and/or gastrointestinal (GI) toxicity of the used anti-tumour drug in patients who consumed such proteins daily over the period of time for which they were subjected to the anti-tumour treatment compared to patients subjected to the same anti-tumour treatment but not consuming the aforementioned proteins.

In one embodiment the aforementioned proteins also satisfy one or more of the parameters listed below:

| | |
|---|---|
| Per 100 g | Per 15 g port. |

| | | | | |
|---|---|---|---|---|
| Energy | 1538 | kJ | 230.7 | kJ |
| | 368 | kcal | 55.2 | kcal |
| Fats | 0 | g | 0 | g |
| of which saturated fats | 0 | g | 0 | g |
| Carbohydrates | 2 | g | 0.3 | g |
| of which sugars | 2 | g | 0.3 | g |
| Proteins | 90 | g | 13.5 | g |
| Salts | 0.2 | g | 0.03 | g |

COMPOSITION IN AMINO ACIDS

| | |
|---|---|
| Alanine | 4.95 g/100 g |
| Arginine | 1.86 g/100 g |
| Aspartic acid | 10.59 g/100 g |
| Cysteine | 2.53 g/100 g |
| Glutamic acid | 16.67 g/100 g |
| Glycine | 1.51 g/100 g |
| Histidine | 1.47 g/100 g |
| Isoleucine | 6.01 g/100 g |
| Leucine | 10.21 g/100 g |
| Lysine | 8.86 g/100 g |
| Methionine | 2.06 g/100 g |
| Phenylalanine | 2.79 g/100 g |
| Proline | 5.05 g/100 g |
| Serine | 4.25 g/100 g |
| Threonine | 6.55 g/100 g |
| Tryptophan | 1.81 g/100 g |
| Tyrosine | 2.77 g/100 g |
| Valine | 5.48 g/100 g |
| TOTAL | 95.42 g/100 g |

The composition as provided is based on the proteins in lyophilised form. In accordance with one embodiment the whey protein concentrate in accordance with any one of the above-listed embodiments is administered to patients subjected to anti-tumour treatment by means of one or more anti-tumour drugs and/or radiotherapy.

In particular, the whey protein concentrate in any of the above-listed embodiments is particularly useful in all of those cases in which said anti-tumour treatment brings about, in the patients to whom it is administered, one or more of the following side effects: malnutrition, haematological toxicity, gastrointestinal toxicity.

As known by a person skilled in the art, these side effects unfortunately are present in any anti-tumour treatment, whether administered by means of chemotherapy, immunotherapy, inhibition of tumour growth, radiotherapy, or any combination thereof. A non-limiting example of chemotherapies according to the present invention is given by 5-fluorouracil, Actinomycin D, Altretamine, Asparaginase, Bendamustine, Bleomycin sulfate, Busulfan, Cabazitaxel, Capecitabine, Carboplatin, Carmustine, Cyclophosphamide, Cisplatin , Citarabine, Chlorambucil, Dacarbazine, Daunorubicin, Docetaxel, Doxorubicin, Doxorubicin hydrochloride, Liposomal doxorubicin, Epirubicin hydrochloride, Eribulin, Estramustine, Etoposide, Everolimus, Fludarabine phosphate, Fluorouracil, Fotemustine, Gemcitabine, Idarubicin hydrochloride, Hydroxyurea, Ifosfamide, Irinotecan hydrochloride, Lomustine, Melphalan, Mercaptopurine, Methotrexate, Mitomycin, Mitoxantrone, Olaparib, Oxaliplatin, Paclitaxel, Pemetrexed, Procarbazine, Raltitrexed, Streptozocin, Tegafur-uracil, Temozolomide, Temsirolimus. Thioguanine, Tiotepa, Topotecan, Treosulfan, Vinblastine sulfate, Vincristine sulfate, Vindesina, Vinflunina, Vinorelbine.

A non-limiting example of immunotherapies according to the present invention is represented by: Brentuximab vedotin, Cetuximab, Edrecolomab, I britumomab, Ipilimumab, Nivolumab, Panitumumab, Pembrolizumab, Pertuzumab, Ramucirumab, Rituximab, Tositumomab, Trastuzumab, and Trastuzumab emtasine.

Inhibitors of tumour growth are drugs that inhibit particular enzymes that allow the growth of a tumour, for example the protein kinase. A non-limiting example of inhibitors of tumour growth according to the present invention is represented by: Afatinib, Axitinib, Bortezomib, Bosutinib, Cabozantinib, Ceritinib, Crizotinib, Dabrafenib, Dasatinib, Erlotinib, Gefitinib, Imatinib, Lapatinib, Nilotinib, Pazopanib, Ponatinib, Regorafenib, Ruxolitinib, Sorafenib, Sunitinib, Trametinib, Vandetanib and Vemurafenib.

As known by a person skilled in the art, anti-tumour therapies are often combined therapies that provide the administration of more than one anti-tumour drug, often belonging to various categories (for example chemotherapeutic drug+immunotherapeutic drug, etc.). The present invention also applies to anti-tumour treatments with combined drugs. In addition, pharmacological therapy can also be combined with radiotherapy. The present invention can be applied in this case as well. The whey protein concentrate according to any one of the above-listed embodiments is preferably administered orally.

For oral administration the whey protein concentrate in any one of the above-described embodiments is provided in lyophilised form and is resuspended in a liquid tolerable by the patient, possibly water, a fruit juice, some kind of milk, a fruit and/or vegetable smoothie, a broth, a soup, a yoghurt, a shake, or any food-based liquid of thicker or thinner consistency, pleasing to the patient.

The whey protein concentrate according to any one of the above-listed embodiments can be administered during the course of the day in one or more doses.

A daily dose indicated for an oncology patient is between 10 and 120 grams per day, also depending on the age, gender, and state of health of the patient. Proceeding already from a daily consumption of 10 grams/day, the beneficial effects described herein were observed on oncology patients subjected to anti-tumour treatment.

The greatest amount consumed consolidates the effects, but not all patients have an appetite sufficient or sustained enough to consume doses of approximately 100 grams of protein per day.

A further daily dose indicated for an oncology patient is between 15 and 60 grams/day, also depending on the age, gender, and state of health of the patient.

In one embodiment the daily dose of the proteins according to the invention is between 20 and 40 grams, and, as mentioned above, can be administered in one or more doses.

In one embodiment the daily dose is 30 grams.

The weights specified above are always based on the proteins as described and claimed in lyophilised form.

According to the invention, the daily dose can be provided to the patient in a single dose, or in two, or in three or more doses.

As already mentioned above, the whey protein concentrate according to the invention is administered to the patient throughout the time for which the patient is subjected to the anti-tumour treatment. In terms of time, this means months or years.

The administration will be performed daily, notwithstanding any indications by the treating doctor or treating medical team. The dose can be adjusted as compared to the daily dose indicated above, depending on the patient. The doctor or the medical team following the oncology patient will be able to regulate the dose of the proteins depending on the response of the patient to the administration thereof.

According to the invention and as discussed in the “Examples” section, the administration of the proteins of the invention in any of their embodiments in combination with the anti-tumour treatment reduces the haematological toxicity and/or gastrointestinal toxicity of the anti-tumour treatment of the oncology patient subjected to said combination compared to oncology patients to whom only the anti-tumour drug is administered.

As is evident from the provided results and from the figures, the haematological toxicity can also be completely eliminated.

The haematological toxicity can be defined in accordance with any parameter normally used by a person skilled in the art in order to assess such toxicity, for example the toxicity can be assessed by means of haematochemical tests, such as one or more of: complete blood count, fibrinogen levels, LDH, alkaline phosphatase, protein electrophoresis, tot/LDL/HDL cholesterol, triglycerides, blood sugar, creatinine, azotaemia, amylase, lipase, insulin levels, cortisol levels, vitamin D, creatine kinase, CRP, ESR, CEA.

Non-limiting examples of haematological toxicity according to the invention which can be observed frequently following anti-tumour treatments are represented by: anaemia, leukopenia, thrombocytopenia, neutropenia, low platelet count, thrombocythemia.

Shifts in the values obtained by one or more of the above-mentioned tests compared to the values measured in the patient before commencement of the anti-tumour therapy (obviously towards abnormal values) are indicative of the haematological toxicity of the therapy.

The values measured by one or more of such tests can present anomalies as compared to the standard values indicated for such tests also before the therapy in the oncology patient. The emergence of further anomalies or a worsening of existing anomalies during the course of the anti-tumour treatment is an indication of the haematological toxicity of the treatment.

The administration of the proteins of the invention in combination with the anti-tumour treatment normally leads to the detection of stable values or even to normalisation thereof in one or more of the above-mentioned tests.

According to the invention and as discussed in the “Examples” section, the administration of the proteins of the invention in any one of their embodiments in combination with the anti-tumour treatment reduces or prevents a worsening of the nutritional state of the oncology patient subjected to said combination as compared to oncology patients to whom only the anti-tumour drug is administered.

According to the invention and as discussed in the “Examples” section, the administration of the proteins of the invention in any of their embodiments in combination with the anti-tumour treatment reduces or

As is evident from the provided results and from the figures, the gastrointestinal toxicity can also be completely eliminated.

The gastrointestinal toxicity can be defined in accordance with any parameter normally used by a person skilled in the art in order to assess such toxicity; for example the emergence, due to the anti-tumour treatment, of one or more of the following: nausea, vomiting, diarrhoea, mucositis, is an indication of a gastrointestinal toxicity of such treatment.

According to the invention, the assessment of the nutritional state of the patient can be performed by means of MNA, MUST, BMA assessment and/or bioimpedance analyses. All of these assessment techniques are commonly known to a person skilled in the art, and the values obtainable by each of the above-mentioned assessments can be considered individually or preferably in combination.

The more accurate are the examinations of the nutritional state of the patient, the better can be the management thereof by the medical team.

For the purposes of the invention, the term “malnutrition” indicates the presence in the patient of sarcopenia and/or cachexia, for example.

As indicated in the summary above, the invention relates to a therapeutic treatment in which the proteins in any of the above-described embodiments are administered to oncology patients subjected to anti-tumour treatment as described and defined above for a prolonged period of time, preferably for the entire period of anti-tumour treatment (including any surgical treatments).

The administration in the method of the invention can be performed in the described ways and with the described dosage in accordance with any of the above-stated embodiments.

For the purposes of carrying out the present invention, said whey protein concentrate is obtainable from a sample of lactoserum by the process described in European patent EP2882305. The process can be applied for example to cow or goat whey or a combination thereof and comprises the following steps:
a) concentrating said sample of lactoserum thus obtaining a protein concentration of between about 150-300 grams/litre;
b) subjecting said lactoserum concentrate to at least one step of diafiltration;
c) diluting said diafiltered protein concentrate thus obtaining a solution having a concentration of proteins in the range of about 50-90 grams/litre;
d) adding to said solution about 5-10 grams of a pyrogenic silica per litre of solution;
e)) adding about 70-300 ml of ethanol 95% v/v, per kilogram of protein in said solution;
f) acidifying the pH of the solution obtained in item e) up to a value of about 4.5-5.0;
g) heating the acidified solution at a temperature between about 55 and 70° C. for at least 20 minutes wherein said temperature is reached within about 30 minutes;
h) cooling the solution to a temperature between about 10 and 20° C.;
i) carrying out a separation of the cooled solution of proteins;
l) bringing the pH of the separate solution to a value of about 5.8-6.8;
m) subjecting the solution obtained in item I) to at least one step of microfiltration to obtain a protein concentrate having a protein concentration of between about 100 and 300 grams/litre,
n) subjecting the protein concentrate to at least one step of diafiltration;
o) drying and/or freeze-drying said concentrated protein, wherein said protein concentrate from lactoserum has a concentration ≤0.5 grams of lactose per kilogram of protein.

Said lactoserum sample can comprise sweet and/or acidic lactoserum.

The concentration in points a) and n) can be performed by means of ultrafiltration. Furthermore, said ultrafiltration can be performed with membranes having a cutoff between about 5000-15000 dalton.

In addition, still for the purposes of preparing the lactoserum proteins as described herein, said at least one step of diafiltration in points b) and n) are at least 3 in number. Furthermore, said at least one step of diafiltration in step b) can be performed using demineralised, deionised water and/or an osmotic solution as solvent.

Such diafiltration can be performed using 3 volumes of said solvent for each volume of the concentrated protein solution.

For the preparation of the whey proteins according to the present description, said step d) can also be performed by adding about 7 gr of fumed silica for each litre of said diluted solution.

Said step e) can be performed by adding about 100-150 ml of said ethanol per kilogram of protein in said solution.

Said step g) can be performed at a temperature of about 66° C.

According to the method described herein, in addition, said at least one step of microfiltration in step m) can be performed using a membrane with a cutoff of about 1.2 micron or 0.6 micron.

The proteins resulting from the above-described process are at least 96% pure and have the composition described above.

The preparation of a protein concentrate according to the method described herein can be performed from a lactoserum sample that can comprise both sweet and acidic lactoserum or that can contain only sweet lactoserum or only acidic lactoserum. Lactoserum useful for the purposes of the present invention can be of any origin, and therefore such lactoersum can be obtained from human, bovine, goat's milk, etc., merely by way of example and in a non-limiting manner.

As indicated above, the method comprises a step of concentrating the starting lactoserum sample which can be performed in accordance with any technique known to a person skilled in the art suitable for this purpose. In a preferred embodiment of the present method, the step of concentration is performed by means of ultrafiltration(s). For example, the ultrafiltration according to step a) as described above is performed by means of the use of membranes having a cutoff between about 10000-15000 dalton. Thus, by way of illustration, membranes with a cutoff of 10000, 11000, 12000, 13000, 14000 and/or 15000 can be used. For the purposes of obtaining a lactoserum with a protein concentration of at least 150 grams/litre, one or more steps of ultrafiltration and/or the use of one or more membranes as described above can be required.

The successive phases or step b) of the method above consists in reducing, to the greatest possible extent, lactose from the concentrated lactoserum in accordance with that described above. The total elimination or the partial reduction of the lactose content can be achieved for example by means of at least one step of diafiltration. In general, diafiltration means the separation of micro solutes from a solution of molecules, in this case proteins, by means of ultrafiltration performed with continuous addition of solvent. For example, the diafiltration according to the present method can be performed using demineralised, distilled water and/or osmotic but not saline solutions as solvent. Merely by way of example, three volumes of demineralised water can be used for each volume of concentrated lactoserum. In one embodiment of the present invention, the steps of diafiltration of the concentrated lactoserum are at least 3 in number and are performed using three volumes of demineralised water for each volume of concentrated lactoserum.

The diafiltrated lactoserum as described above is then characterised relative to the starting lactoserum sample by a reduced lactose concentration and is diluted by the addition of a solvent in order to obtain a solution of diafiltrated lactoserum with a protein content of about 50-90 grams/litre. In particular, this solution can have a protein content of about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 ,81 ,82 , 83, 84, 85, 86, 87, 88, 89, 90 grams/litre. In one embodiment of the invention, the solution of diafiltered lactoserum has a protein concentration of about 70 grams/litre. For example, the dilution of the solution of diafiltered lactoserum is performed by means of the use of demineralised water as solvent.

Step d) of the method described herein comprises the addition to the diluted lactoserum solution of about 5-10 grams, for example 7 grams, of a fumed silica per litre of solution.

Fumes silica is a particular type of silica consisting of microscopic drops of amorphous silica which agglomerate to form tertiary particles having specific chemical/physical characteristics. Fumed silica, which is usually present commercially in the form of a powder, is hydrophilic fumed silica cable of fixing lipoproteins, residual fatty material and/or macromolecules present in a given solution. It is not necessary at this juncture to describe the fumed silica in detail, since it is well known and used in a widespread manner in various technical fields by experts. Merely by way of non-limiting example and for the purposes of the present invention, the fumed silica can be of the Aerosil® type, for example Aerosil® 380 and/or Aerosil® 200.

Following the addition of the silica as mentioned above to the solution, about 70-300 ml of 95% v/v ethanol are added per kilogram of proteins. For example, the amount of ethanol added is about 120 ml per kilogram of proteins and, again by way of example, the ethanol is a food-based type.

Next, in accordance with step f) of the method described herein, the pH of the solution is brought to a value between 4.5 and 5.0, for example by means of the use of HCI. The pH of the solution at the end of step f) can be a pH of 4.6, 4.7, 4.8, 4.9, 5.0; for example the pH will have a value of 4.6.

The acidic solution this obtained is then heated to a temperature between 55 and 70° C. for at least 20 minutes. In particular, this heating temperature can be 55, 56, 57, 58, 59, 60, 61, 62, 63, 64, 65, 66, 67, 68, 69, 70 ° C., for example 66° C. For the purposes of the present invention, the heating just described that consists specifically in increasing the temperature from the initial temperature of the starting lactoserum sample to the above-mentioned temperature of interest must be performed within a period of at least 30 minutes. In particular, the inventors of the present method have found that a quicker heating of the solution results in a loss of the whey protein yield, whereas a slower heating results in an unsatisfactory purification of the whey proteins since proteins not of interest, such as casein, are not eliminated. The heated solution is then cooled to a lower temperature between 10 and 20° C., therefore to a temperature of 10, 11, 12, 13, 14, 15, 16, 17, 18, 19 or 20° C.

This cooled solution is then subjected to separation for the purpose of isolating the portion containing the whey proteins. This step i) of the method described herein can be performed in accordance with any separation/purification method deemed by a person skilled in the art to be suitable for obtaining the protein component from the above solution. Purely by way of example, the separation can be performed continuously, using Alfa Laval or Westfalia separators.

The pH of the separated solution, containing the whey proteins, is then brought to a value between 5.8 and 6.8, for example by the use of NaOH. The pH of the solution for purpose of said step I) can be a pH of 5.8, 5.9, 6.0, 6.1, 6.2, 6.3, 6.4, 6.5, 6.6, 6.7 or 6.8, and for example the pH will have a value of 6.4. In accordance with step m) of the method forming the subject of the present description, the whey protein solution is then subjected to at least one step of microfiltration until a protein concentration having a protein concentration between about 100 and 300 grams/litre is obtained. In one embodiment of the present method the microfiltration is performed with membranes having a cutoff of about 12 microns or 0.6 microns. In particular, at least one first step of microfiltration can be performed, using membranes with a cutoff of 12 microns followed by one or more steps of microfiltration using membranes with a cutoff of 6 microns. For example, the membranes to be used for the microfiltration in the method described herein are organic and/or ceramic membranes.

The microfiltered solution of whey proteins is then subjected to at least one step of diafiltration, which can be performed similarly to that already described for step b) of the method of the present description.

Lastly, the microfiltered and diafiltered solution of lactoserum proteins is dried and/or lyophilised, thus obtaining a concentrate of lactoserum whey proteins.

The concentrate of lactoserum or whey proteins obtainable by means of the method described above is characterised, in particular, by having a concentration ≤0.5 grams of lactose per kilogram of proteins. In addition, the method leads to a concentration of whey proteins in which the purity of the proteins contained therein is at least 96%.

The process described above makes it possible to obtain a concentrate of whey proteins having the following characteristics:

| | | |
|---|---|---|
| Alpha-lactalbumin | 20-28% | by weight |
| Beta-lactalbumin | 32-45% | by weight |
| Seroalbumin | 5.9-7.8% | by weight |
| Immunoglobulins | 3-6% | by weight |
| K Casein | 4-7% | by weight |
| Beta casein | 2-5% | by weight |
| Casein alpha | 2-5% | by weight |
| Lactose | <1% | by weight |
| Fat content | 0% | by weight |

| | |
|---|---|
| Per 100 g | Per 15 g port. |

| | | | | |
|---|---|---|---|---|
| Energy | 1538 | kJ | 230.7 | kJ |
| | 368 | kcal | 55.2 | kcal |
| Fats | 0 | g | 0 | g |
| of which saturated fats | 0 | g | 0 | g |
| Carbohydrates | 2 | g | 0.3 | g |
| of which sugars | 2 | g | 0.3 | g |
| Proteins | 90 | g | 13.5 | g |
| Salts | 0.2 | g | 0.03 | g |

COMPOSITION IN AMINO ACIDS

| | |
|---|---|
| Alanine | 4.95 g/100 g |
| Arginine | 1.86 g/100 g |
| Aspartic acid | 10.59 g/100 g |
| Cysteine | 2.53 g/100 g |
| Glutamic acid | 16.67 g/100 g |
| Glycine | 1.51 g/100 g |
| Histidine | 1.47 g/100 g |
| Isoleucine | 6.01 g/100 g |
| Leucine | 10.21 g/100 g |
| Lysine | 8.86 g/100 g |
| Methionine | 2.06 g/100 g |
| Phenylalanine | 2.79 g/100 g |
| Proline | 5.05 g/100 g |
| Serine | 4.25 g/100 g |
| Threonine | 6.55 g/100 g |
| Tryptophan | 1.81 g/100 g |
| Tyrosine | 2.77 g/100 g |
| Valine | 5.48 g/100 g |
| TOTAL | 95.42 g/100 g |

It is clear that all of the embodiments of the present invention can be carried out with proteins obtained in accordance with the process described above.

### EXAMPLES

### 1. Clinical Study

### 1.1. Plan and Objective of the Study

Based on recent data from the literature and the above-mentioned assessments in respect of the role of malnutrition, sarcopenia and cachexia on the prognosis of a patient suffering from neoplastic disease, a randomised 1:1, single blind placebo-controlled study was performed in order to assess the activity of a supplementation based on highly purified whey protein concentrate as described and claimed in the present description in influencing the state of malnutrition in patients who are candidates for chemotherapy.

The concentrate used in the tests described above was obtained in accordance with the detailed method for purifying and concentrating bovine whey, specified in the detailed description above, and also described and claimed in European patent EP2882305, and has the following composition:

| | | |
|---|---|---|
| Alpha-lactalbumin | 20-28% | by weight |
| Beta-lactalbumin | 32-45% | by weight |
| Seroalbumin | 5.9-7.8% | by weight |
| Immunoglobulins | 3-6% | by weight |
| K Casein | 4-7% | by weight |
| Beta casein | 2-5% | by weight |
| Casein alpha | 2-5% | by weight |
| Lactose | <1% | by weight |
| Fat content | 0% | by weight |

| | |
|---|---|
| Per 100 g | Per 15 g port. |

| | | | | |
|---|---|---|---|---|
| Energy | 1538 | kJ | 230.7 | kJ |
| | 368 | kcal | 55.2 | kcal |
| Fats | 0 | g | 0 | g |
| of which saturated fats | 0 | g | 0 | g |
| Carbohydrates | 2 | g | 0.3 | g |
| of which sugars | 2 | g | 0.3 | g |
| Proteins | 90 | g | 13.5 | g |
| Salts | 0.2 | g | 0.03 | g |

COMPOSITION IN AMINO ACIDS

| | |
|---|---|
| Alanine | 4.95 g/100 g |
| Arginine | 1.86 g/100 g |
| Aspartic acid | 10.59 g/100 g |
| Cysteine | 2.53 g/100 g |
| Glutamic acid | 16.67 g/100 g |
| Glycine | 1.51 g/100 g |
| Histidine | 1.47 g/100 g |
| Isoleucine | 6.01 g/100 g |
| Leucine | 10.21 g/100 g |
| Lysine | 8.86 g/100 g |
| Methionine | 2.06 g/100 g |
| Phenylalanine | 2.79 g/100 g |
| Proline | 5.05 g/100 g |
| Serine | 4.25 g/100 g |
| Threonine | 6.55 g/100 g |
| Tryptophan | 1.81 g/100 g |
| Tyrosine | 2.77 g/100 g |
| Valine | 5.48 g/100 g |
| TOTAL | 95.42 g/100 g |

Patient Selection Criteria:
age >18 years;
PS sec. ECOG: 0-2;
Life expectancy >3 months;
Written informed consent;
Histological neodiagnosis of colorectal adenocarcinoma, stage II, III, IV sec. AJCC;
Candidate patients for chemotherapy in accordance with the pathology in anamnesis (chemotherapy based on 5-fluorouracil).

Exclusion Criteria:
Inability of the patient to follow the study procedures;
Uncontrolled metabolic disorders (diabetes mellitus, dyslipidemia);
Severe hepatic or renal insufficiency (AST or ALT >3 times the normal values, creatinine >2.5 mg/dl);

Active infectious pathology;
Severe psychiatric disorders;
Symptomatic or untreated brain metastases.

The study was performed with the following objectives:
PRIMARY OBJECTIVE:
   Assess the variations in the condition of sarcopenia and in the nutritional state of the patients during the course of chemotherapeutic treatment in the group consuming the protein supplement and in the placebo and identify any differences in the clinical development between the two groups;
SECONDARY OBJECTIVES:
   Describe the correlation present between the nutritional state of the patients under examination and the analysed clinicopathological parameters;
   Identify any link between the nutritional condition of the enrolled patients and the toxicity described in association with the chemotherapeutic treatment assessed according to the Common Terminology Criteria for Adverse Events (CTCAE) v 4.1.

1.2. Materials and Methods

The concentrate as described at point 1.1. is able to provide the patient with a high-quality and digestible protein supplement having excellent nutritional values. In general, soluble whey proteins are rich in similar amino acids, however they have a particular smell and taste; the proteins of the invention are completely odourless and tasteless, with a lactose content less than 2%, more precisely less than 1%, and even less than 0.5% and without casein (thus usable also by subjects who are hypersensitive or intolerant to milk derivatives), and can be diluted in water or in other beverages at ambient temperature.

The control group, instead, was subjected to administration of an isocaloric beverage derived from a mixture of 9 g of inulin and 3 g of potato starch; the compound had the same macroscopic characteristics (colour and consistency) and the same neutral taste as the active compound.

All of the patients enrolled in the protocol were subjected, after 3 and 6 months, to a series of measurements of clinical, anthropometric and laboratory-based nature (complete blood count including white blood cell count, fibrinogen levels, lactic dehydrogenase, alkaline phosphatase, total plasma protein concentration and electrophoretic analysis, lipid profile, blood sugar, renal function indices, amylase and lipase, CRP and ESR inflammation indices, creatine phosphokinase, insulin, cortisol, vitamin D and tumour markers) in order to assess as completely as possible the nutritional profile of the subject. (Table 1) appended below.

In addition, clinicopathological data relating to the neoplastic pathology (location, stage of the tumour, previous surgical intervention) were collated and all of the patients were invited to a nutritional counselling session, during which they were asked to compile a food diary in order to define their dietary habits and to provide simple advice, purely of a dietary nature, in relation to the state of health, disturbances reported by the subject, and the potential presence of enterostomy, but also in relation to physical activity and motricity.

The patients were asked to compile, depending on the established times, the MNA® (mini nutritional assessment by the Nestle nutrition institute, which is a questionnaire known in the literature and used currently in nutritional examinations and matters) in order to assess the presence of malnutrition or the risk that a picture of malnutrition will manifest itself (a score between 24.5 and 30 indicates a good state of nutrition, between 24 and 17.5 a risk of malnutrition, and a result <17 defines a picture of malnutrition) and, in association therewith, based on the history of weight loss in the patient, his/her BMI (Body Mass Index) and the recent clinical history, the MUST (Malnutrition Universal Screening Tool) score was assessed at each meeting, this being a five-phase screening tool for identifying adults who are malnourished, at risk of malnourishment (undernourished) and obese. It also includes management guidelines which can be used to develop a therapeutic programme. A score of 0 indicates a low nutritional risk, 1 a moderate risk, and 2 a high risk, requiring treatment.

A detailed anthropometric assessment was also performed by means of careful measurement of weight, height and relative BMI calculation, knee height, arm circumference, calf circumference, waist circumference and hip circumference. For the most complete definition possible of the patient's body profile, a bioimpedance assessment was also performed at each visit; this is, as already described, a simple non-invasive examination performed with a tool that is easy to use and easily transported, capable of describing the state of hydration of the patient and the distribution of the lean and fat body mass with a rather high sensitivity.

During the second and third meetings, performed at three and six months from the start of the chemotherapy, the adverse effects following the treatment were assessed, defined in accordance with the Common Terminology Criteria for Adverse Events (CTCAE).

1.3. Statistical Analysis

The analysis of the data provided a description of the variability of the clinical parameters considered in the two groups of randomisation. The analyses were also performed depending on the stratification by age, gender, stage and location of the disease, and performance status (PS) according to ECOG and according to the Karnofsky scale (KPS).

Analyses describing the single parameters were used, and univariate and multivariate logistic regression was used for the assessment of the link between nutritional state and clinical outcome in terms of toxicity.

The comparison between the two treatment groups was performed by means of Cox's Hazards Regression model.

1.4. Results

| Arruolamento | Recruitment |
|---|---|
| Selezionati per eleggibilità | Selected for eligibility |
| (n = −47) | (n = 47) |
| Esclusi (n = 7) | Excluded (n = 7) |
| Non rispettavano i criteri di | Do not meet the inclusion |
| inclusion (n = 7) | criteria (n = 7) |
| Non hanno accettato di partecipare | Did not agree to participate |
| (n = 0) | (n = 0) |
| Altri motivi (n = 0) | Other reasons (n = 0) |
| Randomizzati (n = 40) | Randomised (n = 40) |
| Distribuzione | Distribution |
| BRACCIO A (ATTIVO) (n = 21) | GROUP A (ACTIVE) (n = 21) |
| Hanno ricevuto I'intervento | Received the expected surgery |
| previsto (n = 21) | (n = 21) |
| Non hanno ricevuto I'intervento | Did not receive the expected |
| previsto(n = 0) | surgery (n = 0) |
| BRACCIO B (PLACEBO) (n = 19) | GROUP B (PLACEBO) (n = 19) |
| Hanno ricevuto I'intervento | Received the expected |
| previsto (n = 19) | surgery (n = 19) |
| Non hanno ricevuto I'intervento | Did not receive the expected |
| previsto (n = 0) | surgery (n = 0) |
| Analisi dei dati (T0) | Data analysis (T0) |
| T₀: | T₀: |
| Analizzati (n = 21) | Analysed (n = 21) |
| T₀: | T₀: |
| Analizzati (n = 19) | Analysed (n = 19) |
| Follow-Up a 3 mesi | Follow-up at 3 months |
| Persi al follow-up (n = 4) | Lost at follow-up (n = 4) |
| Non aderenti al trattamento | Did not adhere to the treatment |
| (n = 2) | (n = 2) |
| Persi al follow-up (n = 1) | Lost at follow-up (n = 1) |
| Non aderenti al | Did not adhere to the |
| trattamento (n = 1) | treatment (n = 1) |
| Analisi dei dati (T₁) | Data analysis (T₁) |
| T₁: Analizzati (n = 15) | T₁: Analysed (n = 15) |
| Esclusi dall'analisi | Excluded from the analysis |
| (perché persi al | (because lost at follow-up |
| follow-up o non aderenti al | or did not adhere to the |
| trattamento (n = 6) | treatment (n = 6) |
| T₁: Analizzati | T₁: Analysed |
| (n = 17) | (n = 17) |
| Esclusi dall'analisi | Excluded from the analysis |
| (perché persi | (because lost at follow-up or |
| al follow-up o non aderenti al | did not adhere to the |
| trattamento (n = 2) | treatment (n = 2) |

FLOW DIAGRAM SUMMARISING THE TEST PERFORMED

The flow diagram above shows, on the basis of that suggested by the Transparent Reporting of Trials (CONSORT), all the phases (enrolment, distribution, follow-up and data analysis) of the randomised clinical trial described herein.

47 potentially eligible patients were initially selected, of which 7 patients were then excluded because they did not meet the inclusion criteria of our study insofar as they were suffering from gastric neoplasia (n=5) and breast neoplasia (n=2); our assessments were thus performed on a total of 40 patients suffering from colorectal neoplasia: 26 were subjected to surgical treatment and were about to start adjuvant chemotherapy, 14 had metastatic disease from the start.

The patients, who previously had signed inform consent forms, were randomised 1:1 into two groups:
Group A (N=21): active group, intended for consumption of two sachets per day of protein supplement from the start of chemotherapy until its end, for a total of 6 months of treatment;
Group B (N=19): placebo, received administration of a substance based on inulin and potato starch, devoid of active ingredient, to be consumed in 2 sachets per day, from the first day of treatment and for the duration thereof, for a total period of 6 months.

All of the randomised patients received the treatment provided by the protocol.

The patients were assessed at time To, before the start of chemotherapeutic treatment, and were then reassessed at three and six months by a work team formed by oncologists and by a nutritionist.

1.4.1. Basal Assessment

As shown in Table 2 in the appendix below, the enrolled population, randomised into two groups, was fairly homogeneous. With regard to the general characteristics of the patients, the distribution over the two genders was fairly equal, with a total of 15 men and 6 women enrolled in the active group, and 11 men and 8 women in the control group, and also over age, with a median of 65 years in group A and 67 in group B. The locations of the primordial tumour were comparable between the two groups, as was also the subdivision by stage. Considering the comorbidity of the patients, it was possible to show how less than 50% of the patients in the active group, similarly to those in the placebo group, did not have comorbidity, whereas the distribution of the comorbidities of cardiovascular and endocrine-metabolic type was varied. The assessment of the Performance Status, both according to ECOG and according to KPS, demonstrated how the mean value was good in both groups, thus describing a favourable state with regard to activity and ability to perform their separate tasks independently prior to commencement of the treatment.

With regard, instead, to the assessment of the questionnaires completed by the patients in order to assess the nutritional profile, the MNA® demonstrated that 52% of patients of group A and 37% of patients of group B were at risk of malnutrition, a small percentage were already malnourished (14% vs 21%), and the remaining patients (34% vs 42%) were well nourished.

The scores of the MUST described 81% of patients with a score ≥1 (at risk of malnutrition or malnourished) in the active group and 58% in the control group. The analysis of the BMI values, instead, demonstrated an absence, in both groups, of severely underweight patients (BMI ≤18.5 kg/m²), but a fair percentage thereof (38% in the active group and 58% in the placebo group) were overweight or obese (BMI ≥25 kg/m²). The median value for starting body weight was comparable between the two groups.

The measurements performed by bioimpedance analysis revealed a similar distribution of lean and fat mass in the patients of both groups, defining a rather homogeneous starting situation characterised by a picture of reduced representation of muscular mass 969.7% in group A and 67.6% in group B) and increased presence of fat mass in all selected patients (30.3% vs 33.4%).

The assessment of the state of body hydration showed a prevalence of well hydrated patients in the active group (57% vs 37%).

The assessment of the haematochemical examinations did not reveal any significant differences between the two groups; an interesting item of data was the vitamin D value, which was insufficient with values much lower than the normal reference limits, both in the patients in the active group and in those in the placebo group (17.3 vs 15.4).

1.4.2. Reassessment at Three Months

After a period of three months (T₁) from the first meeting, 32 patients were reassessed: 15 from group A and 17 from group B, 21 in adjuvant therapy and 11 with metastatic disease.

Starting from the 40 randomised subjects, 8 therefore were not included in the clinical and instrument-based reassessments at the time T₁ because they were lost at the follow-up or because they did not adhere to the treatment.

With regard to the active group, 4 patients were lost at the follow-up, whereas 2 patients did not adhere to the treatment.

Among the placebo group patients, 1 was lost at the follow-up and 1 showed limited compliance with the treatment.

To summarise, the population on whom the data analysis was performed at 3 months was formed of 32 subjects: 15 in the active group and 17 in the placebo group.

1.4.2.1. Clinicopathological Characteristics

Due to the fact that changes were identified in the total number of patients visited after three months, we deemed it appropriate to reassess, by means of descriptive and comparative analyses, the characteristics of the new population forming the subject of the study in respect of the distribution of gender, age, comorbidity, stage and location of the disease so as to assess any inhomogeneities between the two groups.

As shown in Table 3 in the appendix below, the distribution of some of these characteristics, between the two treatment groups, demonstrated slight changes with regard to the basal assessment: the population maintained its homogeneous character in respect of the distribution by age and comorbidity, whereas a slight inhomogeneity between group A and group B, respectively, was revealed for gender (greater frequency of men in group A), location of disease (greater frequency of disease in the right colon in group A and in the left colon in group B) and stage of disease (prevalence of subjects at stage II in the active group and at stage IV in the placebo group); these differences were not statistically significant.

The assessment of PS according to ECOG demonstrated how the majority of patients in both groups (87% in group A vs 59% in group B) had a value of 0, no subjects in the active group had a PS >1, and values of PS=2 were present only in the placebo group. Moreover, whereas the PS values for group A remained almost unchanged compared to the basal assessments, in group B a worsening of the clinical conditions was confirmed.

1.4.2.2. Anthropometric, Laboratory-Based and Instrument-Based Characteristics

All of the other anthropometric, laboratory-based and instrument-based characteristics of the patients were then examined so as to identify any significant differences between the two groups possibly able to be correlated with the consumption of the protein supplement for a period of three months (Table 3).

The assessment of the MNA® revealed inhomogeneities between the two groups: all of the patients in the active group turned out to be well nourished, whereas 35% of patients in the placebo group turned out to be at risk of malnutrition. This difference between the two treatment groups was statistically significant (p value of 0.01). In addition, no malnourished subjects were revealed to be present in either of the two groups.

The assessment of the scoring obtained from MUST showed that none of the patients in the active group had a score ≥2, 20% had a score=1, and 80% instead had a MUST=0. In the placebo group the distribution was quite different: 12% of patients had a score ≥2, 29% of 1, and 59% of 0. Whereas in group B the distribution of the results of the MUST was stable compared to the basal assessment, it was possible to observe an improvement of the results, at three months, between the patients in group A.

Another difference between the two groups could be observed in the distribution of the BMI values: none of the patients under examination presented a BMI <18.5 kg/m², however a prevalence of patients of normal weight was observed in the active group and a prevalence of obese subjects was observed in the placebo group, the latter percentage of patients being increased compared to the basal assessment. These differences between the two groups, however, were not statistically significant (p value=0.6).

Nevertheless, the body weight value did not show any significant differences between the two treatment groups: the bioimpedance-based measurements revealed a different distribution of body weight in the two groups with a greater median lean mass value in patients in the active group as compared to the placebo group (71.8% vs 63.6%), however this result was not statistically significant (p value=0.07). The fat mass values were also more favourable in the subjects in the active group (27.9% vs 34.8%).

The laboratory analyses did not reveal any statistically significant differences between the two groups, although slight differences in the median values of amylase and creatine kinase could be identified; the presence of extremely reduced vitamin D values in both groups remained, although they were slightly increased as compared to the basal assessments.

1.4.2.3. Toxicity

The assessment of toxicities following chemotherapeutic treatment after three months revealed an interesting difference in the development of the two groups (FIGS. 1 and 2; Table 3). With regard to haematological toxicity (FIG. 1) (anaemia, leukopenia, thrombocytopenia), it was found that 86% of patients in the active group did not present any type of toxicity linked to the treatment, 6% reported a toxicity of grade 1 (1 subject), and 6% of grade ≥2 (1 subject); the difference from the control group was considerable, in fact 71% of these patients presented toxicity, whereas only 29% did not present any type of adverse effect of the haematological type. This difference was statistically significant, with a p value=0.005.

A similar result was observed in the assessment of toxicities of the gastrointestinal group (FIG. 2) (nausea, vomiting, diarrhoea, mucositis): 94% of patients who consumed the proteins did not present any toxicity following the first three months of chemotherapeutic treatment, only 1 subject (6%) presented toxicity of grade 1, and no patients in the active group presented toxicity ≥2. In the group that consumed the placebo, the distribution of the values was quite different: only 29% of the patients did not present any toxicity, whereas 70% presented adverse effects.

This difference was statistically significant with a p value=0.001.

The distribution of toxicities of other types (neuropathies, Hand Foot Syndrome), instead, did not reveal any considerable differences between the two groups.

1.4.2.4. Time Profile

Comparing the measured basal results with those at the time of reassessment at three months, interesting differences between the two treatment groups were revealed in terms of variations in the MNA®, MUST and body weight distribution.

With regard to the MNA®, a distinction was drawn between patients whose score had improved (passing from a lower value range to a higher value range) and those who had remained unchanged or had even worsened (passing from a higher value range to a lower value range) (Table 4).

It was found that in group A 66% of patients revealed an improvement in the score, and in group B, instead, 64% of patients remained stable or even became worse. This difference, however, was not statistically significant (p value=0.07), however the relative risk (RR) was 0.27 (95%IC=0.06-1.17) demonstrating how the consumption of the protein supplement reduces the risk of a worsening of the MNA score of 73%. (FIG. 4).

With regard to the MUST as well, a distinction was drawn between those who had improved their score (passing from a higher value to a lower value) and those who remained unchanged or became worse (passing from a lower value to a higher value). (Table 5).

It was found that 74% of patients in the active group presented an improvement in their score after three months of treatment, whereas in 71% of patients in the placebo group a stability or worsening was observed. This difference was statistically significant (p valu =0.013), the RR was 0.15 (95% IC=0.03-0.71), defining protein integration as a protective factor of 85% as compared to the finding of a stability or worsening of the nutritional risk of the patient according to MUST (FIG. 5).

The results obtained by the bioimpedance analysis revealed that the lean mass of the patients who had consumed the protein-based supplements increased compared to the measurement at time T₀ (71.8% vs 69.7%), whereas the lean mass of the subjects who had consumed the placebo underwent a reduction from 67.6% to 63.6%. The changes in fat mass demonstrated a similar, complementary favourable trend. The changes observed after three months in the patients in the active group was statistically significant (p value=0.013, Z=-2.480). (Table 6) (FIG. 6).

Based on the changes in the lean mass, the patients in each group were subdivided into two groups: patients with median lean mass values that remained stable or increased over time and patients with reduced mass. (Table 7).

With regard to the active group, 86% of the patients maintained a stable lean mass or experienced an increase in lean mass over the course of time, whereas 33% of patients in the placebo group experienced a reduction in lean mass. This difference was not statistically different, and the RR was 0.33 (95% IC=0.05-2.27), and therefore the consumption of the protein supplement decreases the risk of reduction of lean mass by 67%.

Based on the increase in attention in recent years that has bene placed on the problem of assessing the nutritional state, malnutrition, ad sarcopenia in patients suffering from solid neoplasia, the primary objective of the study was that of assessing the changes in the condition of sarcopenia and in the nutritional state of the patients during the course of chemotherapy.

The analyses were performed on a population formed of 40 patients suffering from colorectal neoplasia at stage II, Ill and IV randomised 1:1 in two separate groups: group A, who were provided with a protein-based supplement formed of whey protein concentrate (as defined in the description, in the claims, and under point 1.1 of the “Examples” section) during the course of chemotherapy, and group B, who were given a placebo, with the objective of identifying any differences in the clinical development between the two groups. The study was conducted on a homogeneous population in respect of gender, age, comorbidity, type and stage of neoplasia, and therefore in an objectively analysable manner. This represented an essential factor for the purposes of the analyses of the two treatment groups, considering the impact that many studies in the literature have attributed to non-modifiable factors associated with the patient (gender, age, comorbidity) and to the primordial tumour (location and stage) in influencing the nutritional state of the patient in different ways.

To summarise, it was observed that at the first nutritional visit all of the patients were in a condition of fair well-being with a prevalence of subjects with PS=0, independently of the treatment group; based on such data, it was found that during the course of therapy this situation was maintained only by those patients who had consumed the protein supplement, whereas the general state of a good percentage of the subjects in the placebo group experienced a worsening after the first three months (95% of patients in group B with PS=0 at time T₀ vs 59% at time T₁). This difference, which cannot be attributed to any discrepancies in the state of the patients prior to commencement of the treatment, is correlated with the consumption of the nutritional supplement.

This data appears to be extremely relevant in light of the influence attributed to the PS of the patient in maintaining a suitable dietetic contribution and good tolerance during the course of chemotherapeutic treatment.

At the time of the basal assessment, about 60% of the subjects, both in the active group and in the placebo group, were defined, based on the results of the MNA®, as being at risk of malnutrition or malnourished, very similar results emerging from the MUST as well, which demonstrated a medium-high nutritional risk in both groups. This evidence was in line with that observed in other studies in respect of the prevalence of changes in the nutritional state in patients suffering from neoplastic disease.

The measurements obtained from the bioimpedance analysis before chemotherapeutic treatment showed how the entire population forming the subject of the study presented a similar body weight distribution (the median value of which was comparable between the two groups), characterised by a reduced representation of the lean mass and an elevated fat mass. As described in the literature, this type of body weight distribution defines a picture of sarcopenia, which can manifest itself independently of the fact that chemotherapeutic treatment has been initiated.

At the time of reassessment performed three months after commencement of the chemotherapeutic treatment, changes of differing profiles were observed in the two treatment groups in respect of the clinical characteristics and the nutritional state of the patients under examination.

Firstly, the MNA® score showed that no patients presented a state of malnutrition, independently of group. This piece of information is very interesting and can be explained on the basis of that demonstrated by many studies underlining how nutritional counselling, which is the first therapeutic approach in subjects at risk of malnutrition, and the fact of placing focus on food and diet, has a significant influence on the state of nutrition of patients.

On the other hand, all of the subjects who consumed the protein supplement were found to be in a state of suitable nutritional health, according to MNA®, in contrast to that observed in the placebo group, in which there were 35% of subjects at risk of malnutrition (p value=0.01). This was confirmed by the results of the MUST at time T₁, which revealed 80% of patients with a low risk of malnutrition in the active group and 41% with a moderate/high risk in the placebo group. It is moreover interesting to note how, comparing these results with those obtained at the time T₀, there was a significant improvement of the scope obtained in both questionnaires in the active group, whereas the changes were mainly minimal, or oriented in a worsening direction, in the placebo group. This information on the one hand confirms the fact that the chemotherapeutic treatment can have a negative effect on the nutritional state of oncology patients, as evidenced in many studies, and on the other hand seems to show that the protein supplement according to the present invention can make such an outcome less definitive and can even improve the nutritional state of the patients.

The analyses also revealed a significant discrepancy in the progression of the median lean mass values from the time T₀ (in which a homogeneity of the values in the two groups was observed) to the time T₁ between the two randomisation groups: this tended in fact to increase or remain stable in the active group, whereas its values decreased in the placebo group. Although the difference between the lean mass values between group A and group B (71.8% vs 63.6%) after the first three months of treatment was not statistically significant, this could be attributable to the low numbers in the analysed sample and to a period of observation that was too short to reveal more striking results; in addition, the comparison between the lean mass values measured in group A at the time T₀ and those observed in the same group at the time T₁ (69.7% vs 71.8%) revealed a statistically significant difference, demonstrating how consumption of the protein supplement according to the invention induces a positive trend with regard to an increase in the lean mass, in spite of that which occurs in the group consuming the placebo, in which the median lean mass value reduced from the first to the second control (67.5% vs 63.6%). In fact, a direct comparison of the profile of the percentage values for lean mass in the two groups shows how 86% of the subjects in the active group had, at three months, a stable or increased lean mass, whereas 33% of patients in the placebo group showed a worsening of the picture of sarcopenia.

The analyses performed in respect of the assessment of haematological (neutropenia, anaemia, thrombocytopenia) and gastrointestinal (nausea, vomiting, diarrhoea, mucositis) toxicities associated with the chemotherapy after the first three months of treatment provided very interesting and surprising results. With regard to the haematological toxicities, the data obtained showed that 86% of the subjects in the active group did not manifest any type of toxicity, in spite of that measured in the patients in the placebo group, in which 71% showed toxicity of grade ≥1. Considering, instead, gastrointestinal toxicities, 94% of the patients who had consumed the protein supplement according to the invention did not present any kind of effect, and no patients demonstrated toxicity ≥2, and only 1 patient reported toxicity of grade 1. Among the patients in the placebo group, instead, 59% reported having suffered from toxicity of grade ≥1 during the course of the chemotherapy. The assessment of the neurological toxicities, instead, did not reveal any significant differences between the two treatment groups.

2. Test on Cells

In order to verify the purity and the particularity of the concentrate of the invention, this was assayed on different cell cultures in comparison with the 4 whey protein concentrates, not produced by the applicant, sold the most on the European clinical food market.

The assayed concentrates are indicated by Prot inv (concentrate of the invention as described under point 1.1) and prot. Conc 1, 2, 3 and 4.

Cells Used:
VERO: monkey renal epithelial cells
MDBK: bovine renal epithelial cells
CRFK: cat renal epithelial cells

For all of the cells, the seeding was 40000 cells per millilitre of medium. The count was performed after 72 hours by the same technician using a MALASSEZ cell. The table below shows the number of cells per millilitre of medium after 72 hours of culture. All of the cultures were performed in a culture dish measuring 25 cm² in an incubator with CO₂ atmosphere.

The test was carried out under the following conditions:
Control culture with 10% FCS
Culture with culture medium alone (HAM/F12)
The concentrate of the invention and the 4 commercial concentrates were added to the medium HAM F12 at a concentration of 70 g/litre (protein concentration comparable to FCS.
Separation of trypsin cells/EDTA

| | | | Prot | Prot | Prot | Prot | Prot |
|---|---|---|---|---|---|---|---|
| Cells | SVF10% | HAM/F12 | inv | conc 1 | conc 2 | conc 3 | conc 4 |
| VERO | 280000 | 75000 | 160000 | 2500 | 45000 | 0 | 1200 |
| MDBK | 300000 | 62000 | 170000 | 3000 | 12000 | 0 | 900 |
| CRFK | 260000 | 58000 | 150000 | 500 | 14000 | 0 | 600 |

The results obtained demonstrate that the concentrate of the invention allows good cellular growth and does not show any toxicity.

The concentrates conc n 1, 3 and 4 triggered death of the cells in culture and also presented a strong toxicity, at least for Prot. Conc 3.

Prot. Conc 2 allows slight cell survival.

### Appendices (Tables)

TABLE 1

| | Screening e | | | |
|---|---|---|---|---|
| | Randomisation | | | |
| | (colorectal | T₀ | T₁ | T₂ |
| | adjuvant and | (pre- | (3-4 | (6-7 |
| | metastatic) | therapy) | months) | months) |
| 1st oncology | X | | | |
| visit | | | | |
| (patient | | | | |
| identification: | | | | |
| GLAM | | | | |
| (multidisciplinary | | | | |
| corporate | | | | |
| work group), | | | | |
| department, | | | | |
| 1st central | | | | |
| appointments | | | | |
| visits) | | | | |
| Clinical and | | X | X | X |
| nutritional | | | | |
| examination | | | | |
| Questionnaire n.2 | | X | X | X |
| (MUST, MNA) | | | | |
| BIOIMPEDANCE | | X | X | X |
| ANALYSIS | | | | |
| (sarcopenia) | | | | |
| Haematochemical | X | | X | X |
| examinations | | | | |
| Toxicity | | | X | X |
| according to | | | | |
| CTCAE | | | | |

Clinical and nutritional examination, anthropometric measurements, BMI, PS according to ECOG, KPS.

Haematochemical examinations: complete blood count, fibrinogen levels, LDH, alkaline phosphatase, protein electrophoresis, tot/LDL/HDL cholesterol, triglycerides, blood sugar, creatinine, azotaemia, amylase, lipase, insulin levels, cortisol levels, vitamin D, creatine kinase, CRP, ESR, CEA.

TABLE 2
Basal clinicopathological characteristics of the enrolled patients (N = 40)

| | GROUP | | GROUP | |
|---|---|---|---|---|
| | A | | B | |
| | (N = 21) | | (N = 19) | |
| Gender, n (%) | | | | |
| M | 15 | (71) | 11 | (57) |
| F | 6 | (29) | 8 | (43) |
| Age, mean, years | 65 | | 67 | |
| Location of the tumour, n (%) | | | | |
| Colon right | 11 | (52) | 4 | (22) |
| Colon left | 6 | (29) | 12 | (63) |
| Rectum | 4 | (19) | 3 | (15) |
| Comorbidity, n (%) | | | | |
| none | 9 | (43) | 6 | (31) |
| Cardiovascular (CV) | 5 | (24) | 6 | (31) |
| Endocrine metabolic (EM) | 2 | (9) | 3 | (16) |
| CV + EM | 5 | (24) | 4 | (22) |
| Stage, n (%) | | | | |
| II | 8 | (38) | 6 | (31) |
| III | 6 | (28) | 6 | (31) |
| IV | 7 | (34) | 7 | (38) |
| Performance Status according to ECOG, n (%) | | | | |
| 0 | 18 | (86) | 18 | (95) |
| 1 | 3 | (14) | 1 | (5) |
| Performance Status according | 100 | (90-100) | 100 | (80-100) |
| to KPS, median (range) | | | | |
| MNA, n (%) | | | | |
| 24-30(no malnutrition) | 7 | (34) | 8 | (42) |
| 17-23.5 (risk of malnutrition) | 11 | (52) | 7 | (37) |
| <17 (malnutrition) | 3 | (14) | 4 | (21) |
| MUST, n (%) | | | | |
| 0 | 4 | (19) | 11 | (58) |
| 1 | 9 | (43) | 6 | (32) |
| ≥2 | 8 | (38) | 2 | (10) |
| BMI, n (%) | | | | |
| 18.5-24-9 kg/m² (normal weight) | 13 | (61) | 8 | (42) |
| 25-30 kg/m² (overweight) | 5 | (24) | 7 | (36) |
| 30.1-40 kg/m² (obesity) | 3 | (14) | 4 | (22) |
| <18.5 kg/m² (underweight) | 0 | | 0 | |
| Weight, median, (range), Kg | 72 | (54-96) | 73 | (60-88) |
| Lean mass, median (range), % | 69.7 | (50.4-86) | 67.6 | (50.7-85.6) |
| Fat mass, median (range), % | 30.3 | (14-79.6) | 33.4 | (23-50) |
| Body dehydration, n (%) | | | | |
| No | 12 | (57) | 7 | (37) |
| Si | 8 | (38) | 9 | (47) |
| HB, median, g/dl | 12.8 | (10.3-15.5) | 13.0 | (10.9-14.6) |
| Neutrophils, median (range), | 3.4 | (1.8-14.5) | 4.0 | (1.4-14.8) |
| thousand/mm³ | | | | |
| Lymphocytes median (range), | 1.9 | (0.74-2.9) | 1.9 | (0.2-12.2) |
| thousand/mm³ | | | | |
| Platelets, median (range), | 222 | (117-355) | 257 | (136-600) |
| thousand/mm³ | | | | |
| Fibrinogen, median (range), mg/dl | 365 | (241-595) | 359 | (182-569) |
| LDH, median (range), UI/L | 203 | (131-440) | 237 | (93-375) |
| Alkaline phosphatase, median (range), UI/L | 84.5 | (44.0-208.0) | 84 | (49.0-225.0) |
| Albumin, median (range), g/dl | 4.0 | (3.0-4.6) | 4.0 | (3.0-4.4) |
| Alfa1, median (range), g/dl | 0.3 | (0.19-0.5) | 0.3 | (0.1-0.4) |
| Alfa2, median (range), g/dl | 0.8 | (0.55-1) | 0.8 | (0.6-1.7) |
| Beta1, median (range), g/dl | 0.4 | (0.4-7.8) | 0.5 | (0.4-0.7) |
| Beta2, median (range), g/dl | 0.3 | (0.2-0.7) | 0.4 | (0.3-0.5) |
| Gamma, median (range), g/dl | 1 | (0.5-1.5) | 1 | (0.7-1.5) |
| Total proteins, median (range), g/dl | 6.9 | (6.4-7.8) | 7.0 | (6.4-7.9) |
| Total cholesterol, median (range), mg/dl | 186.5 | (103.0-216.0) | 200.0 | (158.0-262.0) |
| LDL cholesterol, median (range), mg/dl | 118 | (44-155) | 127.5 | (86.0-195.8) |
| HDL cholesterol, median (range), mg/dl | 51 | (33-72) | 48 | (35-89) |
| Triglycerides, median (range), mg/dl | 82.5 | (57.0-158.0) | 86.0 | (58.0-262.0) |
| Blood sugar, median (range), mg/dl | 95 | (80-114) | 95 | (80-192) |
| Creatinine, median (range), mg/dl | 0.8 | (0.4-1.2) | 0.8 | (0.6-1.0) |
| Azotemia, median (range), mg/dl | 24 | (8-71) | 22 | (11-55) |
| Amylase, median (range), UI/l | 59 | (34-128) | 71 | (29-142) |
| Lipase, median (range), UI/l | 32.5 | (14-109) | 29 | (10-80) |
| Cortisol, median (range), nmol/l | 254.1 | (114.0-634.5) | 292.0 | (22.0-388.0) |
| Insulin, median (range), μU/ml | 6.7 | (0.9-23.1) | 7.7 | (4.1-67.2) |
| Vitamin D, median (range), ng/ml | 17.3 | (4.6-34.8) | 15.4 | (5.8-33.2) |
| CK, median (range), UI/l | 59 | (21-108) | 48 | (21-124) |
| C-reactive protein, median | 0.5 | (0.04-8.6) | 0.5 | (0.07-8.7) |
| (range), mg/dl | | | | |
| ESR, median (range), mm/h | 14 | (1.73-48.0) | 14.5 | (2.0-79.0) |
| CEA (range), μU/ml | 3.2 | (0.5-215.3) | 2.3 | (0.5-267.0) |

TABLE 3
Clinicopathological characteristics of the patients assessed at three months (N = 32)

| | GROUP | | GROUP | | |
|---|---|---|---|---|---|
| | A | | B | | P |
| | (N = 15) | | (N = 17) | | value |
| Gender, n (%) | | | | | |
| M | 11 | (73) | 9 | (52) | NS |
| F | 4 | (27) | 8 | (48) | |
| Age, mean, years | 68 | | 67 | | NS |
| Location of the tumour, n (%) | | | | | |
| Colon right | 9 | (60) | 4 | (24) | NS |
| Colon left | 5 | (34) | 10 | (59) | |
| Rectum | 1 | (6) | 3 | (17) | |
| Comorbidity, n (%) | | | | | |
| none | 5 | (33) | 6 | (35) | NS |
| Cardiovascular (CV) | 3 | (21) | 4 | (24) | |
| Endocrine metabolic (EM) | 2 | (13) | 3 | (18) | |
| CV + EM | 5 | (33) | 4 | (23) | |
| Stage, n (%) | | | | | |
| II | 7 | (46) | 6 | (35) | NS |
| III | 4 | (27) | 4 | (24) | |
| IV | 4 | (27) | 7 | (41) | |
| Performance Status according to ECOG, n (%) | | | | | |
| 0 | 13 | (87) | 10 | (59) | 0.2 |
| 1 | 2 | (13) | 6 | (35) | |
| 2 | 0 | | 1 | (6) | |
| Performance Status according | 100 | (70-100) | 100 | (70-100) | 0.2 |
| to KPS, median (range) | | | | | |
| MNA, n (%) | | | | | |
| 24-30 (no malnutrition) | 15 | (100) | 11 | (65) | 0.01 |
| 17-23.5 (risk of malnutrition) | 0 | | 6 | (35) | |
| <17 (malnutrition) | 0 | | 0 | | |
| MUST, n (%) | | | | | |
| 0 | 12 | (80) | 10 | (59) | 0.3 |
| 1 | 3 | (20) | 5 | (29) | |
| ≥2 | 0 | | 2 | (29) | |
| BMI, n (%) | | | | | |
| 18.5-24-9 kg/m² (normal weight) | 6 | (40) | 4 | (24) | 0.6 |
| 25-30 kg/m² (overweight) | 4 | (27) | 6 | (35) | |
| 30.1-40 kg/m² (obesity) | 5 | (33) | 7 | (41) | |
| <18.5 kg/m² (underweight) | 0 | | 0 | | |
| Weight, median, (range), Kg | 77 | (52-98) | 72.5 | (57-96) | NS |
| Lean mass, median (range), % | 71.8 | (55.6-86.5) | 63.6 | (51.1-77.2) | 0.07 |
| Fat mass, median (range), % | 27.9 | (14.4-74.4) | 36.9 | (23.5-42.7) | 0.09 |
| Body dehydration, n (%) | | | | | |
| No | 10 | (67) | 8 | (47) | 0.6 |
| Si | 4 | (27) | 5 | (29) | |
| HB, median, g/dl | 12.8 | (11-16.1) | 12.3 | (8.7-13.9) | 0.1 |
| Neutrophils, median (range), thousand/mm³ | 2.5 | (1.1-6.6) | 2.5 | (0.1-9.1) | 0.6 |
| Lymphocytes median (range), thousand/mm³ | 1.9 | (1.4-2.9) | 1.5 | (1-3.0) | NS |
| Platelets, median (range), thousand/mm³ | 163 | (74-304) | 165 | (84-294) | NS |
| Fibrinogen, median (range), mg/dl | 335 | (139-514) | 351 | (279-484) | NS |
| LDH, median (range), UI/L | 249 | (160-788) | 212 | (168-463) | NS |
| Alkaline phosphatase, median (range), UI/L | 111.5 | (56-154) | 97 | (50-455) | NS |
| Albumin, median (range), g/dl | 3.8 | (3.5-4.4) | 3.8 | (3.1-4.6) | NS |
| Alfa1, median (range), g/dl | 0.3 | (0.2-0.4) | 0.3 | (0.2-0.4) | NS |
| Alfa2, median (range), g/dl | 0.7 | (0.6-0.9) | 0.7 | (0.5-0.9) | NS |
| Beta1, median (range), g/dl | 0.5 | (0.4-0.7) | 0.5 | (0.4-0.7) | NS |
| Beta2, median (range), g/dl | 0.3 | (0.3-0.51) | 0.3 | (0.3-0.5) | NS |
| Gamma, median (range), g/dl | 1.1 | (0.8-1.4) | 0.9 | (0.5-1.5) | NS |
| Total proteins, median (range), g/dl | 6.9 | (6.5-7.3) | 6.8 | (5.7-7.6) | NS |
| Total cholesterol, median (range), mg/dl | 166 | (136-222) | 185 | (89-292) | NS |
| LDL cholesterol, median (range), mg/dl | 95 | (47.8-148) | 105 | (27-183) | NS |
| HDL cholesterol, median (range), mg/dl | 45 | (31-61) | 62 | (6-86) | NS |
| Triglycerides, median (range), mg/dl | 145 | (64-503) | 138 | (54-240) | NS |
| Blood sugar, median (range), mg/dl | 93 | (83-119) | 95 | (88-139) | NS |
| Creatinine, median (range), mg/dl | 0.8 | (0.5-1.4) | 0.8 | (0.6-1.3) | NS |
| Azotemia, median (range), mg/dl | 24.5 | (14-52) | 18.8 | (9-86) | NS |
| Amylase, median (range), UI/l | 57.5 | (29-244) | 87.2 | (45-201) | NS |
| Lipase, median (range), UI/l | 53 | (16-214) | 53 | (18-607) | NS |
| Cortisol, median (range), nmol/l | 251.5 | (202-386) | 241.6 | (115-775) | NS |
| Insulin, median (range), μU/ml | 8.8 | (2.5-20.7) | 11 | (5-53.6) | NS |
| Vitamin D, median (range), ng/ml | 18.5 | (11-31) | 14.1 | (4.5-30.8) | NS |
| CK, median (range), UI/l | 58 | (24-142) | 87.5 | (32-290) | NS |
| C-reactive protein, median (range), mg/dl | 0.4 | (0.14-7) | 0.4 | (0.09-6.1) | NS |
| ESR, median (range), mm/h | 16.5 | (5-68) | 13 | (4-70) | NS |
| CEA (range), μU/ml | 2.8 | (0.5-10.2) | 2.9 | (0.86-67.3) | NS |
| Haematological toxicity | | | | | |
| 0 | 13 | (86) | 5 | (29) | 0.005 |
| 1 | 1 | (6) | 8 | (47) | |
| ≥2 | 1 | (6) | 4 | (23) | |
| Gastrointestinal toxicity | | | | | |
| 0 | 14 | (94) | 5 | (29) | 0.001 |
| 1 | 1 | (6) | 4 | (23) | |
| ≥2 | 0 | | 8 | (47) | |
| Other toxicities (HFS neuropathies) | | | | | |
| 0 | 6 | (40) | 7 | (41) | 0.9 |
| 1 | 6 | (40) | 7 | (41) | |
| ≥2 | 3 | (20) | 3 | (18) | |

TABLE 4
MNA assessment comparison (n = 32)

| MNA score from | GROUP A | | GROUP B | | RR | |
|---|---|---|---|---|---|---|
| T₀ to T₁ (%) | (N = 15) | | (N = 17) | | (95% IC) | P value |
| Improved | 10 | (66) | 6 | (35) | 0.27 | 0.07 |
| Unchanged/ | 5 | (33) | 11 | (64) | (0.06-1.17) | |
| Worse | | | | | | |

TABLE 5
MUST assessment comparison (n = 32)

| MUST score from | GROUP A | | GROUP B | | RR | |
|---|---|---|---|---|---|---|
| T₀ to T₁ (%) | (N = 15) | | (N = 17) | | (95% IC) | P value |
| Improved | 11 | (74) | 5 | (29) | 0.15 | 0.013 |
| Unchanged/ | 4 | (26) | 12 | (71) | (0.03-0.71) | |
| Worse | | | | | | |

TABLE 6
Lean mass measurement comparison in
the two groups at time T₀ and T₁

| | | | | | Z | |
|---|---|---|---|---|---|---|
| | | | | | (Wilcoxon | P |
| | Time T₀ | | Time T₁ | | test) | value |
| Group A | 69.7 | (50.4-86) | 71.8 | (55.6-86.5) | −2.480 | 0.013 |
| Group B | 67.5 | (50.7-85.6) | 63.6 | (51.1-85.2) | −0.178 | 0.859 |

TABLE 7
Lean mass development comparison

| Lean mass from T₀ | GROUP A | | GROUP B | RR | |
|---|---|---|---|---|---|
| to T₁ (median), (%) | (N = 14) | | (N = 12) | (95% IC) | P value |
| Increased/ | 12 | (86) | 8 (66) | 0.33 | 0.250 |
| Unchanged | | | | | |
| Reduced | 2 | (14) | 4 (33) | (0.05-2.27) | |

1. A method of inhibiting tumor growth comprising administering to a subject in need thereof a whey protein concentrate in combination with one or more anti-tumor drugs and/or radiotherapy;
wherein said whey protein concentrate has the following composition

| | | |
|---|---|---|
| Alpha-lactalbumin | 20-28% | by weight |
| Beta-lactalbumin | 32-45% | by weight |
| Seroalbumin | 5.9-7.8% | by weight |
| Immunoglobulins | 3-6% | by weight |
| K Casein | 4-7% | by weight |
| Beta casein | 2-5% | by weight |
| Casein alpha | 2-5% | by weight |
| Lactose | <1% | by weight |
| Fat content | 0% | by weight. |

2. The method of claim 1, wherein said concentrate is administered to patients subjected to anti-tumour treatment with one or more anti-tumour drugs and/or radiotherapy.

3. The method of claim 1, wherein said anti-tumour treatment causes in patients to whom it is administered one or more of the following side effects: malnutrition, haematological toxicity, gastrointestinal toxicity.

4. The method of claim 1, wherein said anti-tumour drugs are chemotherapeutics, immunotherapeutics, tumour growth inhibitors, or a combination thereof,

5. The method of claim 4 wherein said anti-tumour drugs are 5-fluorouracil, Actinomycin D, Altretamine, Asparaginase, Bendamustine, Bleomycin sulphate, Busulfan, Cabazitaxel, Capecitabine, Carboplatin, Carmustine, Cyclophosphamide, Cisplatin , Citarabine, Chlorambucil, Dacarbazine, Daunorubicin, Docetaxel, Doxorubicin, Doxorubicin hydrochloride, Liposomal doxorubicin, Epirubicin hydrochloride, Eribulin, Estramustine, Etoposide, Everolimus, Fludarabine phosphate, Fluorouracil, Fotemustine, Gemcitabine, Idarubicin hydrochloride, Hydroxyurea, Ifosfamide, Irinotecan hydrochloride, Lomustine , Melphalan, Mercaptopurine, Methotrexate, Mitomycin, Mitoxantrone, Olaparib, Oxaliplatin, Paclitaxel, Pemetrexed, Procarbazine, Raltitrexed, Streptozocin, Tegafur-uracil, Temozolomide, Temsirolimus. Thioguanine, Tiotepa, Topotecan, Treosulfan, Vinblastine sulfate, Vincristine sulfate, Vindesina, Vinflunina, Vinorelbine, Brentuximab vedotin, Cetuximab, Edrecolomab, Ibritumomab, Ipilimumab, Nivolumab, Panitumumab, Pembrolizumab, Pertuzumab, Ramucirumab, Rituximab, Tositumomab, Trastuzumab, Trastuzumab emtasine, Afatinib, Axitinib, Bortezomib, Bosutinib, Cabozantinib, Ceritinib, Crizotinib, Dabrafenib, Dasatinib, Erlotinib, Gefitinib, Imatinib, Lapatinib, Nilotinib, Pazopanib, Ponatinib, Regorafenib, Ruxolitinib, Sorafenib, Sunitinib, Trametinib, Vandetanib, or Vemurafenib.

6. The method of claim 1, wherein said whey proteins are administered at a daily dosage ranging from 10 to 120 grams.

7. The method of claim 6 wherein said whey concentrate is administered at a daily dosage of between 15 and 80 grams.

8. The method of claim 7 wherein said concentrate is administered at a daily dosage of between 20 and 40 grams.

9. The method of claim 8 wherein said concentrate is administered at a daily dosage of 30 grams.

10. The method of claim 5, wherein said concentrate is administered in one, two or three doses during the day.

11. The method of claim 1, wherein the administration of said concentrate in combination with the anti-tumour treatment reduces the haematological toxicity and/or the gastrointestinal toxicity and/or the malnutrition detectable in the oncological patient treated with said combination with respect to that detectable in cancer patients treated only with the anti-tumour therapy.

12. The method of claim 1, wherein the administration of said concentrate in combination with the anti-tumour treatment reduces or prevents malnutrition in the cancer patient treated with said combination with respect to cancer patients treated only with the anti-tumour therapy.

13. The method of claim 12 in said malnutrition is represented by sarcopenia and/or cachexia.

14. The method of claim 11, wherein said whey protein concentrate is obtainable from a sample of lactoserum by a process comprising the following steps:
a) concentrating said sample of lactoserum thus obtaining a protein concentration of between about 150-300 grams/liter;
b) subjecting said lactoserum concentrate to at least one step of diafiltration;
c) diluting said diafiltered protein concentrate thus obtaining a solution having a concentration of proteins in the range of about 50-90 grams/liter;
d) adding to said solution about 5-10 grams of a pyrogenic silica per liter of solution;
e) adding about 70-300 ml of ethanol 95% v/v, per kilogram of protein in said solution;
f) acidifying the pH of the solution obtained in item e) up to a value of about 4.5-5.0;
g) heating the acidified solution at a temperature between about 55 and 70° C. for at least 20 minutes wherein said temperature is reached within about 30 minutes;
h) cooling the solution to a temperature between about 10 and 20° C.;
i) carrying out a separation of the cooled solution of proteins;
l) bringing the pH of the separate solution to a value of about 5.8-6.8;
m) subjecting the solution obtained in item 1) to at least one passage of microfiltration to obtain a protein concentrate having a protein concentration of between about 100 and 300 grams/liter,
n) subjecting the protein concentrate to at least one step of diafiltration;
o) drying and/or freeze-drying said concentrated protein, wherein said protein concentrate from lactoserum has a concentration ≤0.5 grams of lactose per kilogram of protein.
